(19) United States
(12) Patent Application Publication
(10) Pub. No.: US 2024/0306310 A1
(43) Pub. Date: Sep. 12, 2024

(54) IMPLANTABLE MEDICAL DEVICE CASE FIT WITH SELECTIVE ENCAPSULATION

(71) Applicant: Cardiac Pacemakers, Inc., St. Paul, MN (US)

(72) Inventors: Benjamin J. Haasl, Forest Lake, MN (US); Katherine Provo, Plymouth, MN (US); James Michael English, Cahir (IE); Moira B. Sweeney, St. Paul, MN (US)

(73) Assignee: Cardiac Pacemakers, Inc., St. Paul, MN (US)

(21) Appl. No.: 18/597,007

(22) Filed: Mar. 6, 2024

Related U.S. Application Data
(60) Provisional application No. 63/450,491, filed on Mar. 7, 2023.

(51) Int. Cl. H05K 3/28; A61N 1/375; A61N 1/378; H01M 50/202; H01M 50/242; H01M 50/247; H01M 50/284; H05K 5/06; H05K 1/18

(52) U.S. Cl. CPC H05K 3/284; A61N 1/3758; A61N 1/378; H01M 50/202; H01M 50/242; H01M 50/247; H01M 50/284; H05K 5/065; H05K 5/069; H01M 2220/30; H05K 1/18; H05K 2201/10037; H05K 2203/1316

(57) ABSTRACT

Methods of manufacturing a medical device, and medical devise made by such methods. One or more layers or portions of encapsulant are used to secure components of a medical device in a housing. The case fit-up of the device may be modified to reduce reliance on size tolerances of components of the medical device, while still accounting for any anticipated changes in component size due to aging.

### CROSS REFERENCE TO RELATED APPLICATIONS

The present application claims the benefit of and priority to U.S. Prov. Pat. App. No. 63/450,491, filed Mar. 7, 2023, the disclosure of which is incorporated herein by reference.

### BACKGROUND

A wide variety of active implantable medical devices (IMD) are known, including pacemakers, defibrillators, neural modulation systems, drug pumps, circulation systems, etc. Such devices often include an enclosure, often made of metal and hermetically sealed, which contain operational circuitry for the active IMD. The operational circuitry may be secured or fixed within the housing to prevent relative motion, which can cause damage to electrical interconnections and/or componentry. Prior approaches generally have used a frame and/or motion limiting inserts to hold the operational circuitry in desired positions. Prior approaches have to include accommodations for changes in components over time as, for example, the batteries used in an implantable medical device may change shape as the battery ages. For example, a frame and/or spacers may be provided along with air gaps between components to allow for fit-up, swelling, shifting, etc. of various components. The provided frame, spacers, and gaps need to account for variation as well in the actual dimensions of components which can vary in thickness/height dimensions by 1% or more. When stacked one on top of another, the total variation that a particular design needs to accommodate can make manufacturing and canister sealing processes/designs difficult. New and alternative solutions are desired.

### OVERVIEW

The present inventors have recognized, among other things, that a problem to be solved is the need for new and/or alternative ways to ensure appropriate case fit-up inside an implantable medical device canister/housing. The goal may be achieved by having first and second encapsulant layers provided in the device, which are molded on and/or adjacent to components of the device. The encapsulant layers can reduce any need to account for component tolerances when designing device size, as the tolerances merely effect how much of the encapsulant will be used, rather than the size of the encapsulant.

A first illustrative, non-limiting example takes the form of a method of assembling an implantable medical device comprising: preparing a printed circuit board assembly (PCBA) including a plurality of PCBA mounted first components; using a first mold, applying a first encapsulant layer to at least a portion of at least one of the plurality of PCBA mounted first components, without encapsulating the PCBA entirely; electrically connecting a plurality of second components to the PCBA after the first encapsulant layer is applied to the PCBA; with the first encapsulant layer and PCBA adjacent a first canister portion, and using a second mold, applying a second encapsulant layer over at least a portion of at least one of the second components; placing a second canister portion adjacent at least part of the second encapsulant layer; and welding the first canister portion relative to the second canister portion.

Additionally or alternatively, at least one of the second components is a battery having first and second faces and surrounding edges, the first and second faces being of larger area than the surrounding edges, and the method comprises leaving a gap in the second encapsulant layer to create a space around a face of the battery. Additionally or alternatively, the battery comprises at least one battery cell, the battery cell having a beginning of life thickness with a BOL tolerance, and an end of life thickness which is greater than the beginning of life thickness, the thickness of the battery cell being defined in a direction between the first and second faces of the battery, further wherein the gap is sized to accommodate a change in thickness of the battery cell between beginning and end of life thicknesses, but does not account for the BOL tolerance of the battery.

Additionally or alternatively, the second encapsulant layer also at least partly encapsulates at least one of the PCBA mounted first components. Additionally or alternatively, the second encapsulant layer also contacts the first encapsulant layer. Additionally or alternatively, the first and second encapsulant layers each define one or more air gaps relative to at least the first and second canister portions, wherein the air gaps are sized to accommodate changes in size of one or more of the first components and second components, but are not sized to account for size tolerances of the first components and second components. Additionally or alternatively, the method includes placing the first canister portion adjacent to the first encapsulant layer after the first encapsulant layer is applied. Additionally or alternatively, the method includes securing the first canister portion to the first encapsulant layer during the step of applying the first encapsulant layer. Additionally or alternatively, the first canister portion is used along with the first mold when the first encapsulant layer is applied during the step of encapsulating at least a portion of at least one of the PCBA mounted first components in the first encapsulant layer. Additionally or alternatively, the method includes attaching a telemetry coil to the PCBA after the step of encapsulating at least a portion of at least one of the PCBA mounted first components in the first encapsulant layer.

Another illustrative and non-limiting example takes the form of an implantable medical device comprising: a housing having a first portion and a second portion; a printed circuit board assembly (PCBA) including a plurality of PCBA mounted components; a battery comprising at least one battery cell, the battery cell having a beginning of life thickness with a BOL tolerance, and an end of life thickness which is greater than the beginning of life thickness; and an encapsulant surrounding at least portion of the battery, the encapsulant being placed by molding over at least a portion of the battery cell, wherein the encapsulant defines an air gap sized to account for the difference between the battery cell beginning of life thickness and the battery cell end of life thickness, wherein the air gap is not sized to account for the BOL tolerance of the battery cell.

Additionally or alternatively, the encapsulant comprises a first portion and a second portion, the first portion of the encapsulant being attached to the first portion of the housing during a first molding step. Additionally or alternatively, the second portion of the encapsulant is attached to the first portion of the encapsulant during a molding step, and is not attached to the second portion of the housing. Additionally or alternatively, the second portion of the encapsulant is attached to the first portion of the encapsulant during a molding step, and is attached to the second portion of the housing by an adhesive. Additionally or alternatively, the housing is electrically conductive.

Another illustrative and non-limiting example takes the form of a method of manufacturing an implantable medical device having a housing having an internal dimension along a first direction, and a plurality of components that align in the first direction, the method comprising: determining, for each of the plurality of components that align in the first direction, a maximum change in size along the first direction during an expected life of the implantable medical device, and summing the maximum change for each component to determine a tolerance; designing a gap to be placed between the housing and an encapsulant layer to be applied to the plurality of components as a mechanical support for the plurality of components, such that the gap exceeds the tolerance in a dimension of the first direction; forming the encapsulant layer; and closing the housing around the components, the encapsulant layer, and the gap; wherein the gap is designed without accounting for variation in size or placement of the components.

Additionally or alternatively, the housing comprises at least first and second housing pieces, and step of forming the encapsulant layer is performed by placing the encapsulant layer against an interior side of the first housing piece. Additionally or alternatively, the step of forming the encapsulant layer comprises the use of a mold to define the gap. Additionally or alternatively, the step of forming the encapsulant layer includes forming a first portion of the encapsulant layer in a first forming step, and forming a second portion of the encapsulant layer in a second forming step. Additionally or alternatively, at least one of the components is a battery having a thickness which increases as the battery ages and depletes.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative prior art implantable medical device;

FIG. 2 is a block process flow diagram;

FIGS. 3-7 illustrate steps for assembling an implantable medical device; and

FIG. 8 shows in graphical form a case stack fit-up activity.

### DETAILED DESCRIPTION

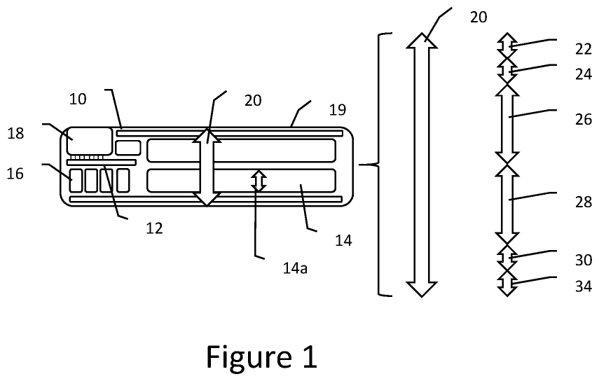

FIG. 1 shows an illustrative prior art implantable medical device. The device 10 may contain a number of components, such as a printed circuit board assembly (PCBA) 12 which includes a circuit board and a number of electrical components thereon, while also coupling to other components. The electrical components may include, for example and without limitation, a microprocessor, microcontroller, application specific integrated circuit (ASIC), amplifiers, various electric chips, crystal oscillators, antennae, inductors, resistors, capacitors, discrete or on-chip logic circuits, memory, etc. The device 10 may further include, for example, batteries 14 and larger size capacitors 16, feedthrough assemblies 18, and any other suitable devices. If the device is an electrical therapy device (a stimulator, pacemaker, defibrillator, etc.) it may only have electric components; mechanical components may also be included for example with drug pumps or heart assist products (LVAD, for example). Speakers and transducers may be included as well, if desired.

With all the various components in the device stack-up or case fit-up is a design consideration. Fit-up is part of the design process in which the dimensions of the outer canister 19 is determined. A minimum size is desirable for patient comfort and to reduce, for example, the size of a pocket in which the device is placed and to reduce the size of incisions used during surgery. However, the thickness 20 of the device housing 19 has to ensure that all the components can fit properly using worst case dimensions. Thus, for example, the thickness 20, as shown to the right side of FIG. 1, includes considering the nominal thicknesses 26, 28 of components, maximum variance or tolerances 24, 30 of the nominal thicknesses 26, 28, and worst-case changes in thickness 22, 34.

Changes due to aging can occur with any component. Some battery cells, such as certain Lithium batteries, are known to swell from beginning of life to end of life. Such battery swelling may be observed along the major faces of the battery—that is, as shown in FIG. 1, the battery 14 has minor faces on the left and right sides, and would have major faces on top and bottom in the view as shown. Swelling may be observed in the direction shown at 14a. Such swelling can be modeled using a battery aging test, and worst-case changes in thickness 22, 34 can be developed.

Failure to properly account for tolerances and any potential changes due to aging can lead to various device failures. One failure is the inability to properly close and seal the device during manufacturing. A more serious failure would be breach of hermeticity if swelling occurs due to aging, or failure of a weld seam or cracking of a housing wall due to pressure present at the time of case welding. If the hermetic seal on the device is lost, an electrical failure due to fluid ingress may occur and/or potentially worse issues for the patient as some materials in the device may be harmful to human health.

The present inventors have identified the use of an encapsulant or dampening layer as a potential solution to these issues, easing the design process by making case fit-up simpler. A discussion of process flow, an illustration of assembly steps, and an example of simplified case fit-up analysis follows.

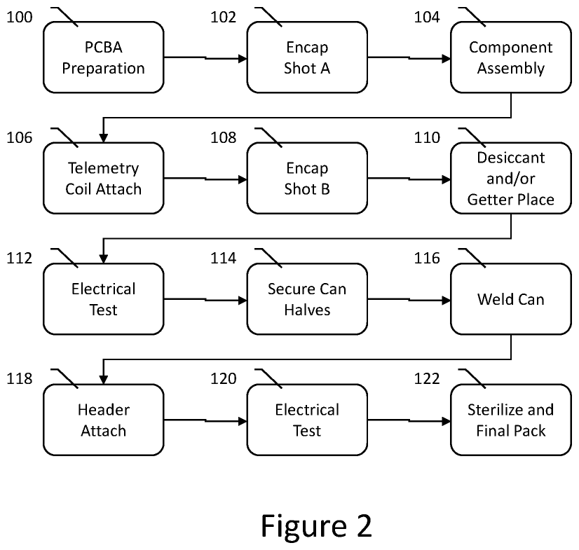

FIG. 2 is a block process flow diagram. The example shown does not include all process steps, and instead highlights those steps that are closest in time and/or most pertinent to the encapsulant layer application and case fit-up process. At 100, a printed circuit board assembly (PCBA) is prepared. This may include manufacture of the printed circuit board (PCB) with traces, through holes, and desired shape/size, followed by pick, place and solder processes for the application of components (such as those noted previously) on the PCB, forming a PCBA. Typically, one or more bake-out steps are performed to remove residues and/or reduce strain/stress forces, as needed. Next, a first layer or portion of encapsulant (“Shot A”) is applied at 102. Step 102 may occur before or after placement of the PCBA in a housing or canister portion, so that the encapsulant is applied using the housing or canister as a portion of a mold for such placement. Other molds, spacers, mandrels and the like may be used to provide desired shape, gaps, spacing, etc. in the resultant encapsulant. For example, gaps may be provided for thermal isolation or thermal conductivity, or to allow manufacturing processes to be completed, as noted in U.S. patent application Ser. No. 18/432,570, filed Feb. 5, 2024, titled IMPLANTABLE MEDICAL DEVICE WITH MOTION DAMPING LAYER, the disclosure of which is incorporated herein by reference.

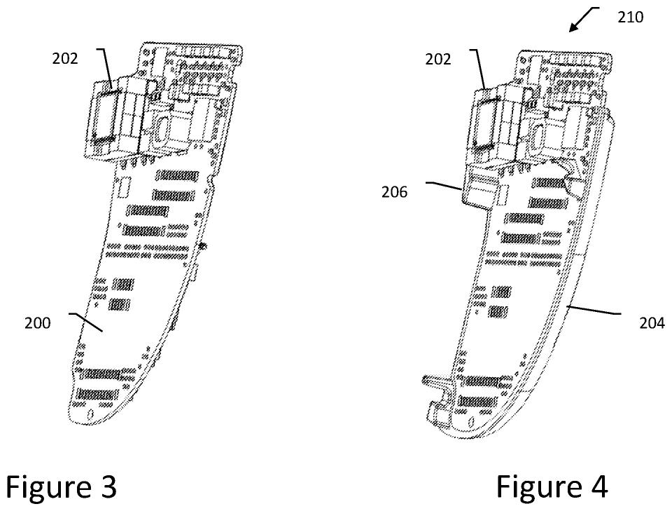

In an example, further component assembly occurs as indicated at 104. This may include the placement of larger components in relation to the PCBA, such as device batteries and/or other use-specific components, such as the relatively large capacitors used with an implantable defibrillator, recharging circuitry used with a rechargeable neuro-stimulator, and/or drug reservoir and/or pump mechanisms for an implantable drug delivery system. One example noted in FIG. 2, which is present in the example of FIGS. 3-7, is attachment of a telemetry coil 106 (a step which may be omitted if the telemetry coil is omitted or is located elsewhere, such as in the device header, for example).

A second layer or portion of encapsulant is then applied, as indicated at 108. In some examples, a single encapsulant layer/portion is provided, and one of steps 102 or 108 may be omitted if desired. Again, a mold as well as spacers, mandrels, etc. may be used to obtain desired shape, gaps, spacing, etc. in the resultant encapsulant. In some examples, at least one portion of the device housing or canister may be present in block 108, and molding in either of steps 102 or 108 may include injecting and attaching the encapsulant to the inner surface of the device housing or canister, as desired and suitable to a particular build. Other examples may use a mold only, and do not allow the encapsulant to become attached/molded to the inner surface of the device housing or canister. A desiccant and/or getter (such as a hydrogen getter) can then be placed as indicated at block 110; block 110 may be moved up or down in the process steps, as desired. Desiccant and/or getter may instead be provided in the encapsulant itself, if desired, such that block 110 becomes a part of one or both of blocks 102 and/or 108.

Electrical testing may then be performed, as indicated at 112. It is common for electrical testing to be performed at various build levels, to capture any electrical failures caused by previous processing steps, or due to component and/or connection failures, before incurring the cost of added components and process steps. Thus, an electrical test as indicated at 112 may be performed earlier and/or later in the process. A test performed at the point shown, 112, would occur after the device has been coupled to internal batteries and would be performed to ensure, for example, that circuitry of the device receives appropriate power levels from the device battery and can perform a range of functions that are then available. Earlier in the process, for example, after step 100, an externally powered electrical test may be performed, as desired. Testing at 112 may include testing the telemetry operations, as block 112 is shown subsequent to block 106. Other tests may include testing that initial, manufacturing, or production firmware has been loaded correctly, that battery voltage can be measured/monitored, and/or that electrical or other outputs can be generated and controlled appropriately. Calibration of components and/or firmware can be tested at block 112 as well.

If electrical testing is passed 112, the partially built IMD proceeds to later steps. In this example, the next step is to secure can halves, as indicated at 114. Block 114 refers to the “can” or canister/housing, and “halves,” which may not necessarily be actual halves; the intent is to indicate that a two-part housing is being secured together relative to the contents, including at least the PCBA of block 100, components of blocks 104, 106 and 110, and encapsulant of blocks 102 and 108. The can portions, once placed and secured in block 114, are then welded together at 116. Any suitable weld process can be used; a common approach is to perform laser welding of housing portions together. The device header is then attached, as indicated at 118, which generally completes the mechanical process for most IMD builds. Electrical testing is performed again, as indicated at 120; this last test may include uploading production firmware to replace manufacturing firmware and/or upgrading to a latest version of IMD firmware, as well as again confirming device functionality following assembly and welding steps. A lead (or dummy lead having electrical connections but terminating in a test fixture) may be inserted in the device header for testing at 120. The completed assembly is then passed on to sterilization and final pack processes, as indicated at 120.

FIGS. 3-7 illustrate steps for assembling an implantable medical device. As side/perspective view of a PCBA is shown at 200, with a number of components thereon, including a relatively thicker component at 202, which may be, for example, a transformer (the example shown being for an implantable defibrillator that uses a transformer for DC:DC voltage step up, as is known in the art).

As shown at FIG. 4, a selective encapsulant layer has been applied, with one portion thereof at 204 on the “back” side of the PCBA, providing, to some extent, anti-vibration shielding for the circuitry components on the back side of the PCBA. Another portion of the encapsulant layer extends as shown at 206 and partly surrounds the thick component 202, proving mechanical shielding for further manufacturing steps. Any variance in the placement or size of the thick component 202 can be absorbed by the encapsulant layer portion at 206, as dimensions of the resultant layer 206 are determined by the mold used for placing it, rather than by the thick component 202.

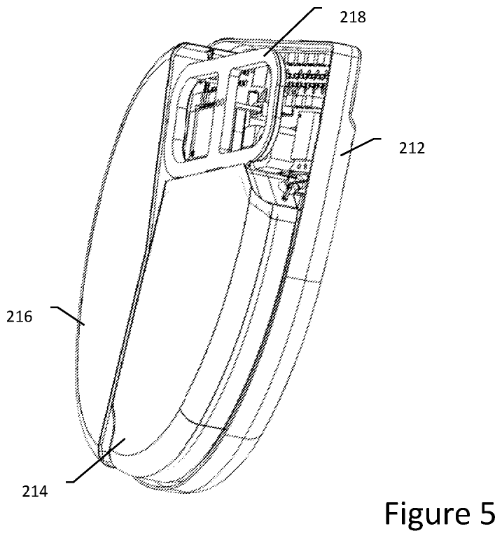

FIG. 5 illustrates placement of a first housing portion at 212. Portion 212 may be provided instead in FIG. 4, and may be used in place of a mold to determine the outer shape of the first encapsulant layer portion at 204 (see FIG. 4). Any variation in the positioning and/or size of components on the “back” side of the PCBA, relative to the housing portion 212 are masked by the use of the encapsulant layer, which has outer dimension determined by the mold used in its placement, or by the first housing portion 212. This may provide greater flexibility in the placement of such components, and simplifies the build process. At most, a few shims may be used to ensure adequate fit-up between the encapsulant 204 and the housing portion 212 (assuming it is not used as the mold for placement of one or more sides of the encapsulant layer). The use of the encapsulant minimizes variation in the stackup of the various components.

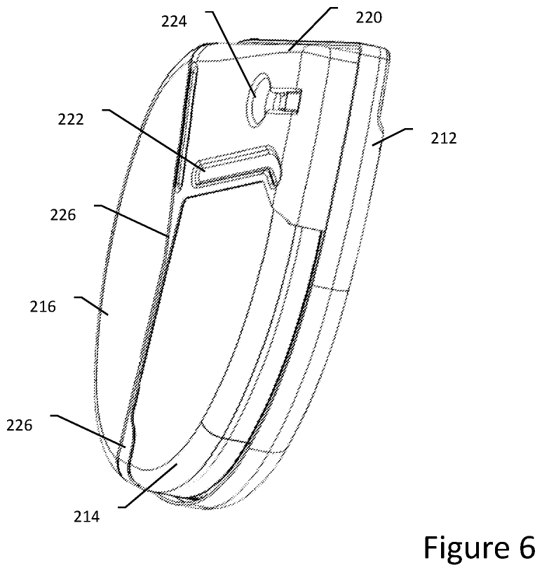

Further components are added, as shown in FIG. 5, in which a battery stack 214 and capacitor stack 216 have been added. By “stack,” the intended meaning is that at least one, and sometimes two or three of the same component type are provided adjacent or “stacked” upon one another. A telemetry coil 218 is also placed, as shown. Next, a second encapsulant layer 220 portion is added, as shown in FIG. 6. Here, the encapsulant portion 220 is applied using a mold, which defines a channel 222 that acts to reduce thermal conduction to the header region of the battery 214, and a void 224 which is provided for use during residual gas analysis (RGA) testing. The channel 222 and void 224 are further discussed in U.S. patent application Ser. No. 18/432,570, filed Feb. 5, 2024, titled IMPLANTABLE MEDICAL DEVICE WITH MOTION DAMPING LAYER, the disclosure of which is incorporated herein by reference.

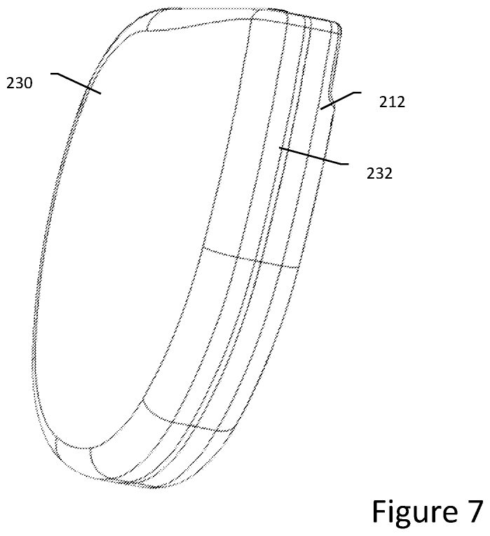

In the example shown, the second portion of encapsulant 220 is applied using a mold that also defines edges next to the battery 214 and capacitor 216, so that the thickest portion of the assembly shown in FIG. 6 is the encapsulant 220. That is, the encapsulant layer at 224 and at 226, when created, extends beyond the adjacent portions of one or more components, such as the battery 214. This leaves a gap which, when the second portion of the canister 230 is added as shown in FIG. 7, will allow for expansion of the battery during the life of the device without applying stress to two housing portions 212, 230 and/or the weld 232 that joins them.

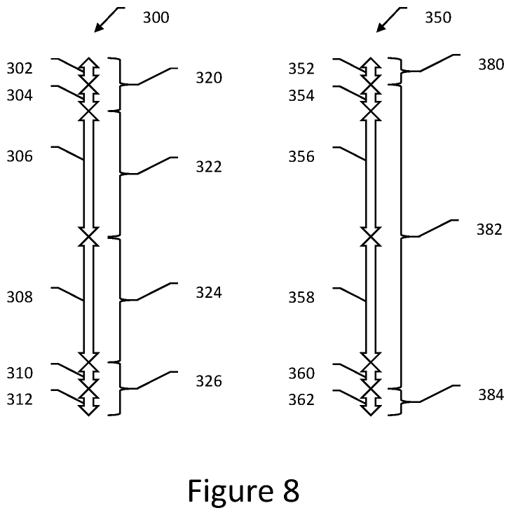

FIG. 8 shows in graphical form a case stack fit-up activity. At 300 is an example of the type of analysis that would be done with a conventional approach. A plurality of components 306, 308 are included in the case stackup. For component 306, changes due to aging of the component are accounted for with a first variance 302, and differences in placement and size is accounted for with a second variance 304, resulting in the total tolerance 320 needed for the first component 306, relative to the nominal thickness 322 of the first component 306. That is, the tolerance 320 is the required “empty” space needed in the stackup calculation to ensure that the maximum thickness of component 306 can be accommodated in the IMD housing. Similar analysis takes place for second component 308, having nominal thickness 324, differences in placement and size is accounted for with a third variance 310, and changes due to aging at 312, yielding second tolerance 326.

At 350 a different approach is illustrated. When using an encapsulant approach, the nominal thicknesses of components 356, 358 and the variances due to differences in placement and size of those components, shown at 354 and 360, are all accommodated within the thickness of the one or more encapsulant layers, as indicated at 382. This thickness 382 is a process parameter controlled by the size of the mold or molds used when the encapsulant is placed, rather than being subject to variances of multiple components and placement of those components. The potential changes in component thickness over the duration of component life, shown at 352 and 362 remain to be accounted for in the stack fit-up activity, and result in spaces shown at 380, 384. The fit-up activity is simplified to obtaining estimates of maximum component size changes during component life, and accounting for such changes when designing the molds to ensure adequate space is left inside the device housing to prevent excess strain on the housing and/or welds.

FIG. 8 thus illustrates steps of a method of manufacturing an implantable medical device having a housing having an internal dimension along a first direction, and a plurality of components that align in the first direction. Here the first direction is the direction from top to bottom of FIG. 8, which may correspond to a thickness, width, depth, diameter, diagonal, or other internal dimension of an implantable medical device housing. The method then includes determining, for each of the plurality of components that align in the first direction, a maximum change in size, such as shown at 352 and 362 along the first direction during an expected life of the implantable medical device, and summing the maximum change for each component to determine a tolerance, where the tolerance would be the sum of 380 and 384. Next, in the method, one would design a gap to be placed between the housing and an encapsulant layer to be applied to the plurality of components as a mechanical support for the plurality of components, such that the gap exceeds the tolerance 380+384 in the first direction. Next, the method includes forming the encapsulant layer, such as by insert or injection molding using a mold and/or the housing. In an example, optionally, a spacer, spacers, mandrel or mandrels may be placed against the housing, and molding can take place against the spacer/mandrel and the housing, such that gaps and/or ridges are defined, without separately using a mold for one or more sides of the housing. The encapsulant layer may be formed in one, two or more injection/insertion steps, using as many molds, spacers and/or mandrels (or other mechanics) as are needed to obtain a desired shape. Next, the method includes closing the housing around the components, the encapsulant layer, and the gap, such as by welding. It can be observed that in this method, the gap is designed without accounting for variation in size or placement of the components, that is, variances 354 and 360 can be ignored. One or more of the components may be a battery that swells or otherwise changes shape or size, such as by increasing thickness, as the battery ages and/or becomes depleted.

Each of these non-limiting examples can stand on its own, or can be combined in various permutations or combinations with one or more of the other examples. The above detailed description includes references to the accompanying drawings, which form a part of the detailed description. The drawings show, by way of illustration, specific embodiments. These embodiments are also referred to herein as “examples.” Such examples can include elements in addition to those shown or described. The present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of assembling an implantable medical device comprising:
preparing a printed circuit board assembly (PCBA) including a plurality of PCBA mounted first components;
using a first mold, applying a first encapsulant layer to at least a portion of at least one of the plurality of PCBA mounted first components, without encapsulating the PCBA entirely;
electrically connecting a plurality of second components to the PCBA after the first encapsulant layer is applied to the PCBA;
with the first encapsulant layer and PCBA adjacent a first canister portion, and using a second mold, applying a second encapsulant layer over at least a portion of at least one of the second components;
placing a second canister portion adjacent at least part of the second encapsulant layer; and
welding the first canister portion relative to the second canister portion.

2. The method of claim 1, wherein at least one of the second components is a battery having first and second faces and surrounding edges, the first and second faces being of larger area than the surrounding edges, and the method comprises leaving a gap in the second encapsulant layer to create a space around a face of the battery.

3. The method of claim 2, wherein the battery comprises at least one battery cell, the battery cell having a beginning of life thickness with a BOL tolerance, and an end of life thickness which is greater than the beginning of life thickness, the thickness of the battery cell being defined in a direction between the first and second faces of the battery, further wherein the gap is sized to accommodate a change in thickness of the battery cell between beginning of life thickness and end of life thickness, but does not account for the BOL tolerance of the battery.

4. The method of claim 1, wherein the second encapsulant layer also at least partly encapsulates at least one of the PCBA mounted first components.

5. The method of claim 1, wherein the second encapsulant layer also contacts the first encapsulant layer.

6. The method of claim 1, wherein the first and second encapsulant layers each define one or more air gaps relative to at least the first and second canister portions, wherein the air gaps are sized to accommodate changes in size of one or more of the first components and second components, but are not sized to account for size tolerances of the first components and second components.

7. The method of claim 1, further comprising placing the first canister portion adjacent to the first encapsulant layer after the first encapsulant layer is applied.

8. The method of claim 1, further comprising securing the first canister portion to the first encapsulant layer during the step of applying the first encapsulant layer.

9. The method of claim 8, wherein the first canister portion is used along with the first mold when the first encapsulant layer is applied during the step of encapsulating at least a portion of at least one of the PCBA mounted first components in the first encapsulant layer.

10. The method of claim 1, further comprising attaching a telemetry coil to the PCBA after the step of encapsulating at least a portion of at least one of the PCBA mounted first components in the first encapsulant layer.

11. An implantable medical device comprising:
a housing having a first portion and a second portion;
a printed circuit board assembly (PCBA) including a plurality of PCBA mounted components;
a battery comprising at least one battery cell, the battery cell having a beginning of life thickness with a BOL tolerance, and an end of life thickness which is greater than the beginning of life thickness; and
an encapsulant surrounding at least portion of the battery, the encapsulant being placed by molding over at least a portion of the battery cell, wherein the encapsulant defines an air gap sized to account for the difference between the battery cell beginning of life thickness and the battery cell end of life thickness, wherein the air gap is not sized to account for the BOL tolerance of the battery cell.

12. The implantable medical device of claim 11, wherein the encapsulant comprises a first portion and a second portion, the first portion of the encapsulant being attached to the first portion of the housing during a first molding step.

13. The implantable medical device of claim 12, wherein the second portion of the encapsulant is attached to the first portion of the encapsulant during a molding step, and is not attached to the second portion of the housing.

14. The implantable medical device of claim 12, wherein the second portion of the encapsulant is attached to the first portion of the encapsulant during a molding step, and is attached to the second portion of the housing by an adhesive.

15. The implantable medical device of claim 12, wherein the housing is electrically conductive.

16. A method of manufacturing an implantable medical device having a housing having an internal dimension along a first direction, and a plurality of components that align in the first direction, the method comprising:
determining, for each of the plurality of components that align in the first direction, a maximum change in size along the first direction during an expected life of the implantable medical device, and summing the maximum change for each component to determine a tolerance;
designing a gap to be placed between the housing and an encapsulant layer to be applied to the plurality of components as a mechanical support for the plurality of components, such that the gap exceeds the tolerance in a dimension of the first direction;
forming the encapsulant layer;
closing the housing around the components, the encapsulant layer, and the gap;
wherein the gap is designed without accounting for variation in size or placement of the components.

17. The method of claim 16, wherein the housing comprises at least first and second housing pieces, and step of forming the encapsulant layer is performed by placing the encapsulant layer against an interior side of the first housing piece.

18. The method of claim 16, wherein the step of forming the encapsulant layer comprises the use of a mold to define the gap.

19. The method of claim 16, wherein the step of forming the encapsulant layer includes forming a first portion of the encapsulant layer in a first forming step, and forming a second portion of the encapsulant layer in a second forming step.

20. The method of claim 16, wherein at least one of the components is a battery having a thickness which increases as the battery ages and depletes.
